(19) United States
(12) Patent Application Publication
(10) Pub. No.: US 2014/0052134 A1
(43) Pub. Date: Feb. 20, 2014

(54) LIMB LENGTHENING APPARATUS AND METHODS

(71) Applicant: Bruce Orisek, Aptos, CA (US)

(72) Inventor: Bruce Orisek, Aptos, CA (US)

(21) Appl. No.: 13/763,058

(22) Filed: Feb. 8, 2013

Related U.S. Application Data
(60) Provisional application No. 61/596,564, filed on Feb. 8, 2012.

(51) Int. Cl. A61B 17/72

(52) U.S. Cl. CPC A61B 17/7216
USPC 606/63

(57) ABSTRACT

Embodiments of the invention include implantable apparatus including a magnet and related methods that can be used to lengthen the limbs of a subject. In an embodiment, the invention includes a limb lengthening apparatus including a magnet and a piston having a lengthwise axis, the magnet oriented so as to cause movement of the piston along its lengthwise axis in response to application of an external magnetic field. The apparatus also includes a first body member and a second body member, the first body member and second body member configured to be disposed lengthwise within the bone of a limb to be lengthened. The apparatus also includes a lengthening mechanism, the mechanism including a drive nut, wherein sequential application and removal of the external magnetic field causes the drive nut to rotate around the lengthwise axis of the piston causing the distance between the first body member and the second body member to increase. Other embodiments are also included herein.

This application claims the benefit of U.S. Provisional Application No. 61/596,564, filed Feb. 8, 2012, the contents of which are herein incorporated by reference.

### FIELD OF THE INVENTION

The present invention relates to apparatus and methods that can be used to lengthen the limbs of a subject. More specifically, the present invention relates to an implantable apparatus including a magnet and related methods that can be used to lengthen the limbs of a subject.

### BACKGROUND OF THE INVENTION

There are many conditions wherein it can be desirable to lengthen the limb of a patient. In some cases, lengthening a limb can be used to solve a limb length discrepancy issue. In other cases, lengthening multiple limbs can be used to address issues such as short stature. Leg length discrepancies and/or short statute may arise from many different causes including birth defects, improper bone growth, malnutrition, disease, or trauma.

Some limb lengthening techniques require that the bone of the limb be cut, called an osteotomy or corticotomy. The bone begins development of a callus at this location. The two bone portions are then pulled apart by a mechanical device. This procedure is called a distraction, in which the callus is stretched, thereby lengthening the bone.

Many current mechanical devices used for limb lengthening include external fixators transcutaneously connected to the bone using wires, pins, or screws. Unfortunately, these methods can cause complications such as infections at the points of the transcutaneous connections, discomfort in wearing the fixator for the patient, and the unattractive appearance of the fixator.

Some of the problems associated with external fixators can be avoided by implanting an internal fixation device to perform the distraction. However, internal devices suffer from other issues including complicated implantation procedures and difficulties with initiating and controlling distraction.

### SUMMARY OF THE INVENTION

Embodiments of the invention include implantable apparatus including a magnet and related methods that can be used to lengthen the limbs of a subject. In an embodiment, the invention includes a limb lengthening apparatus including a magnet and a piston having a lengthwise axis, the magnet oriented so as to cause movement of the piston along its lengthwise axis in response to application of an external magnetic field. The apparatus also includes a first body member and a second body member, the first body member and second body member can be configured to be disposed lengthwise within the bone of a limb to be lengthened. The apparatus also includes a lengthening mechanism, the mechanism including a drive nut, wherein sequential application and removal of the external magnetic field causes the drive nut to rotate around the lengthwise axis of the piston causing the distance between the first body member and the second body member to increase.

In an embodiment, the invention includes a method. The method can include implanting a limb lengthening apparatus into the subject the limb lengthening apparatus, applying an external magnetic field to the magnet; and removing the external magnetic field to the magnet.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is an exploded schematic view of a limb lengthening apparatus in accordance with various embodiments.

FIG. 2 is a schematic view of a lengthening mechanism in accordance with various embodiments.

FIG. 3 is an exploded schematic view of portions of a linear to rotational conversion mechanism in accordance with various embodiments herein.

FIG. 4 is a schematic diagram of how some portions of the linear to rotational conversion mechanism interact during operation.

FIG. 5A shows a distal portion of a lengthening apparatus in cross-section, in accordance with various embodiments herein.

FIG. 5B shows a medial portion of a lengthening apparatus in cross-section, in accordance with various embodiments herein.

FIG. 5C shows a proximal portion of a lengthening apparatus in cross-section in accordance with various embodiments herein.

FIG. 6A shows a distal portion of the lengthening apparatus of FIG. 5A in cross-section and rotated by 90 degrees around the lengthwise axis, in accordance with various embodiments herein.

FIG. 6B shows a medial portion of a lengthening apparatus of FIG. 5B in cross-section and rotated by 90 degrees around the lengthwise axis in accordance with various embodiments herein.

FIG. 6C shows a proximal portion of a lengthening apparatus of FIG. 5C in cross-section and rotated by 90 degrees around the lengthwise axis in accordance with various embodiments herein.

FIG. 7 shows an exemplary limb lengthening apparatus disposed within the femur of a patient.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

As described above, there are various issues that can be addressed by lengthening the limb or limbs of a patient. Embodiments herein include an implantable lengthening apparatus than can include a magnet. The application of an external magnetic field operates on the magnet to create a motive force that can be utilized to incrementally lengthen the apparatus in order to provide distraction in which the callus is stretched, thereby lengthening the bone.

In specific, the magnet can be coupled (directly or indirectly) to an apparatus which converts and uses the linear motive force generated in the direction of the lengthwise axis of the limb into rotation of a drive nut around a threaded rod causing the overall length of the apparatus to increase. Each cycle of application of the magnetic field and then removal of the magnetic field results in a defined amount of rotation of the drive nut about the threaded rod and therefore a defined amount of lengthening of the apparatus and the limb in which it is disposed. In various embodiments, the apparatus and the limb in which it is disposed is lengthened 1 mm per on/off cycle of the external magnetic field.

The external magnetic field can be generated in various ways. In some embodiments, an electromagnet can be activated in order to generate the external magnetic field. In some embodiments, a superconducting electromagnet can be used similar to those used in MRI machines. The strength of a magnetic field generated can vary depending the size of the magnet within the limb lengthening apparatus, the size of the bone to be lengthened, the strength of the springs within the limb lengthening apparatus, etc. However, in some embodiments, the magnetic field is between 0.001 and 3.0 Tesla. In some embodiments, the magnetic field is between 0.1 and 3.0 Tesla.

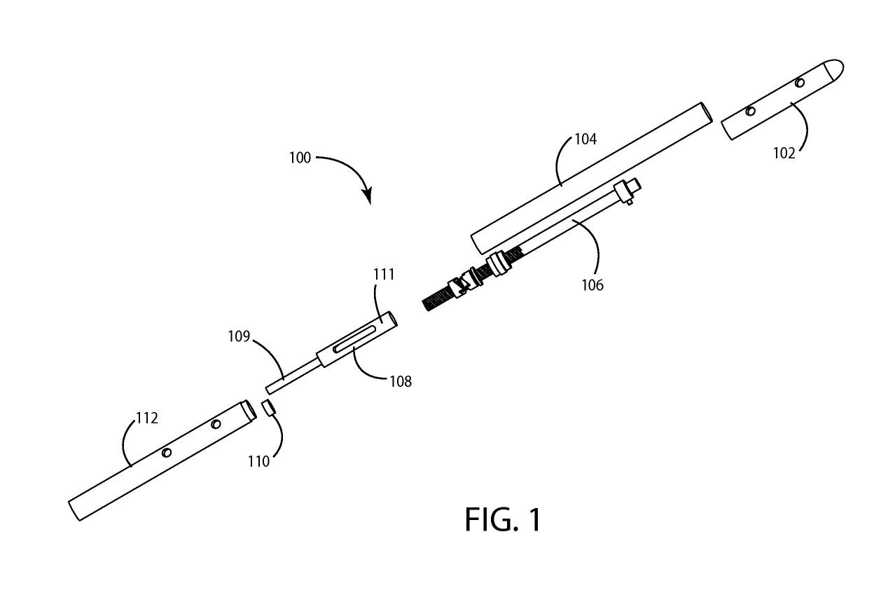

Referring now to FIG. 1, an exploded schematic view of a limb lengthening apparatus 100 in accordance with an embodiment herein is shown. The apparatus 100 can include a plurality of body members. In this embodiment, the apparatus 100 can include a first body member 104 and a second body member 112.

In some embodiments, the apparatus can also include a bullet (or superior-most) 102. The bullet 102 can include a rounded surface on its proximal most end. In some embodiments, the bullet 102 can be coupled to the first body member 104. For example, the bullet 102 can be coupled to the first body member 104 by way of a threaded connection with threads on one component and a complementary portion to engage the threads on the other portion. In some embodiments, the bullet 102 and the first body member 104 are integrated into a single body member.

The apparatus 100 can include a lengthening mechanism 106. The apparatus can further include a magnet 110 and a piston 108. The piston can have a first portion 109 have a first outside diameter and a second portion 111 with a second outside diameter that is larger than the first outside diameter. The magnet 110 can be coupled, directly or indirectly, to the piston 108. This piston can be biased linearly with a spring. For example, the piston can be biased with a spring in a direction opposite to the direction in which the magnet will cause the piston to move when an external magnetic field is applied. As such, the linear force provided by the magnet can overcome the force of the spring. In some embodiments, the magnet 110 can be toroidal in shape. In other embodiments, the magnet 110 can have other shapes. The magnet can be made of any suitable material having sufficient magnet properties. In various embodiments, the magnet is a permanent magnet. In some embodiments, the magnet can include, but it not limited to, ferrite, alnico, and or rare earth materials such as samarium-cobalt and neodymium-iron-boron.

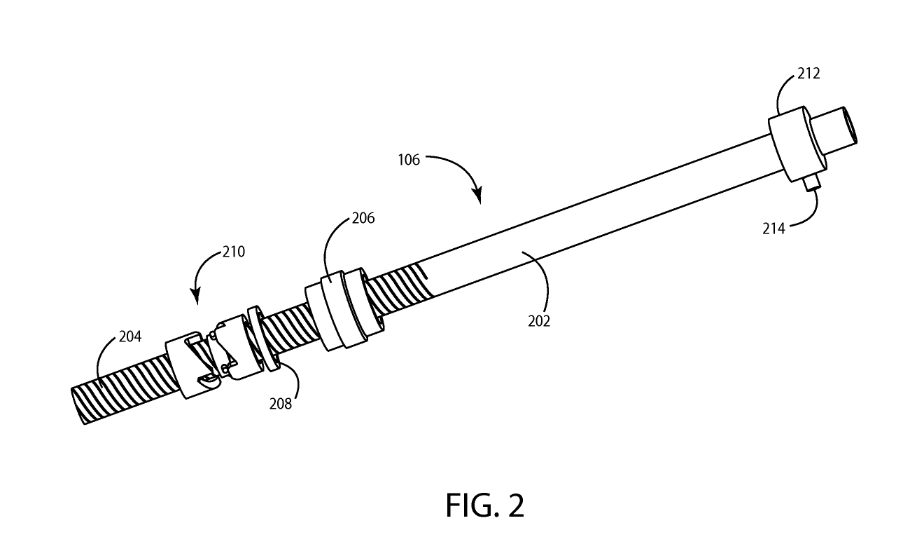

Referring now to FIG. 2, a schematic view of a lengthening mechanism 106 is shown in accordance with an embodiment herein. The lengthening mechanism 106 can include a threaded drive shaft 202. The threaded drive shaft 202 can include a threaded portion 204. The second portion 111 of the piston 108 can have an outside diameter that is larger than the outside diameter of the threaded portion 204 of the threaded drive shaft 202. The piston 108 can include a central lumen into which at least a portion of the threaded portion 204 of the threaded drive shaft 202 can fit. In various embodiments, the threaded drive shaft 202 can include a central channel (not shown in this view) running down the length of the shaft. At the proximal end of the lengthening mechanism 106, a proximal collar 212 can be coupled to the threaded drive shaft 202. An anti-rotation set screw 214 can serve to couple the proximal collar 212 to the threaded drive shaft 202. The set screw 214 can also engage a channel on the first body member 104 preventing the threaded drive shaft 202 from rotating. The lengthening mechanism 106 can also include a junction fitting 206. The junction fitting 206 can be configured to fit over the threaded drive shaft 202 and between the first body member 104 and the second body member 112. When the first body member 104 and second body member 112 move apart from one another as the overall device lengthens, the junction fitting 206 stays with the first body member 104 and moves away from the second body member 112.

The lengthening mechanism 106 can also include a thrust bearing 208. The junction fitting 206 and the thrust bearing 208 can be made of various materials such as biocompatible metals, polymers, ceramics, glasses, or the like. The lengthening mechanism 106 can further include a linear to rotational conversion mechanism 210.

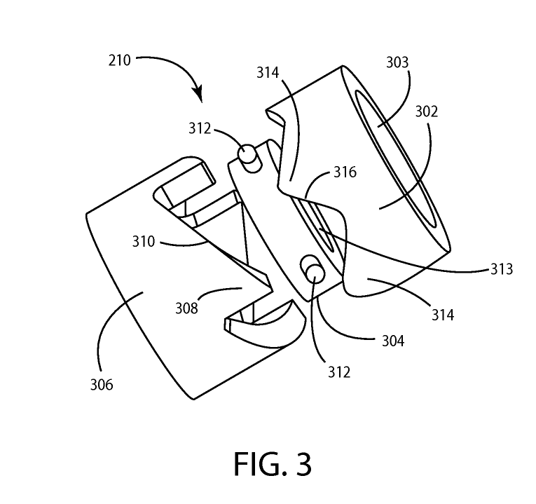

Referring now to FIG. 3, an exploded schematic view is shown of portions of a linear to rotational conversion mechanism 210 in accordance with various embodiments herein. The conversion mechanism 210 can include a first split cam half 302, a drive nut 304, and a second split cam half 306. The first split cam half 302 can include a central channel 303 and a plurality of teeth 314 (or lobes). The number of teeth can vary. In some embodiments, the first split cam half 302 can include from 1 to 20 teeth. In some embodiments, the first split cam half 302 can include from 2 to 6 teeth. In a particular embodiment the first split cam half 302 can include 4 teeth. The teeth can be disposed equidistantly around the circumference of the first split cam half 302. The teeth 314 can each include a bearing surface 316. The drive nut 304 can include a central threaded channel 313, and a plurality of drive pegs 312 (or radial projections). In some embodiments, the drive nut 304 can include from 1 to 20 drive pegs 312. In some embodiments, the drive nut 304 can include from 2 to 6 drive pegs. In a particular embodiment the drive nut 304 can include 4 drive pegs. The drive pegs can be disposed equidistantly around the circumference of the drive nut 304. The second split cam half 306 can include a central channel, a plurality of teeth 308 (or lobes). The number of teeth can vary. In some embodiments, the second split cam half 306 can include the same number of teeth as the first split cam half 302. The teeth 308 can each include a bearing surface 310.

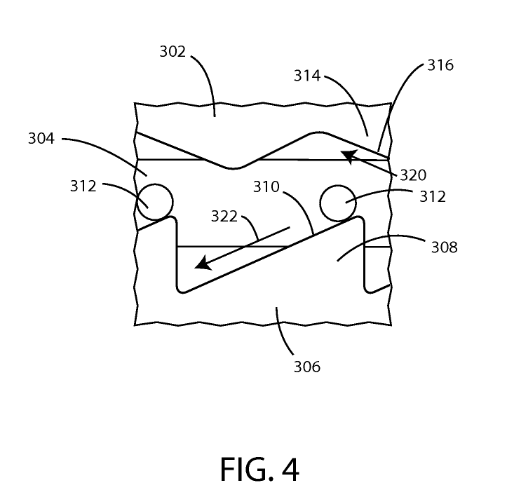

Referring now to FIG. 4, a schematic view is shown of how some portions of the linear to rotational conversion mechanism 210 function. As the second split cam half 306 is pulled downward based on the force provided by the magnet when the external magnetic field is applied (first movement phase), the drive peg 312 contacts the tooth 314 of the first split cam half 302. Specifically, the bearing surface 316 of the first split cam half 302 contacts the drive peg 312. The drive peg 312 moves in the direction of arrow 320 relative to the first split cam half 302, effectively sliding across the bearing surface 316 and causing the drive nut 304 to rotate. Then, when the external magnetic field is withdrawn (second movement phase), based on the force of a spring, the tooth 308 of the second split cam half 306 contacts the drive peg 312. Specifically, the bearing surface 310 of the second split cam half 306 contacts the drive peg 312. The drive peg 312 moves in the direction of arrow 322 relative to the second split cam half 306, effectively sliding across the bearing surface 310 and causing the drive nut 304 to rotate around the lengthwise axis of the apparatus. At the end of the second movement phase, the drive peg 312 comes to a rest disposed in the valley between the teeth of the second split cam half 306. The teeth of the first split cam half 302 and the teeth of the second split cam half 306 are out of phase in that the peaks of the teeth 314 of the first split cam half 302 do not precisely align with the valleys between the teeth 308 of the second split cam half 306.

It will be appreciated that arrows 322 and 320 are simply provided for explanation and represent movement relative to either the first or second split cam half. Actual movement of the drive peg 312 is substantially perpendicular to the lengthwise axis of the apparatus. The total amount of rotation of the drive nut 304 after both phases of movement is dependent on the total number of teeth on the first and second split cam halves. By way of example, when the first and second split cam halves include four teeth each, then the total rotation of the drive nut 304 after both phases of movement can be equal to 90 degrees.

The threads on the interior of the drive nut 304 that contact the threaded drive shaft 202 can be such that after both sequential movement phases of a particular cycle (e.g., wherein a cycle refers to application of magnetic field and then withdrawal of magnetic field), the rotation of the drive nut 304 around the threaded drive shaft causes the drive nut 304 to move a specific distance downward along the lengthwise axis of the threaded drive shaft 202, thereby lengthening the entire limb lengthening apparatus. The entire apparatus is lengthened because the drive peg(s) 312 of the drive nut 304 rests against the valley between teeth of the second split cam half 306 when no magnetic field is applied (resting state). In this position, the second split cam half 306 pushes against the piston which in turn pushes indirectly against the second body member 112. Therefore, because the drive nut has rotated down toward the distal end of the drive shaft 202, the second body member 112 has been effectively pushed away from the first body member 104. In some embodiments, both sequential movement phases of a particular cycle (e.g., application of magnetic field and then withdrawal of magnetic field) cause the limb lengthening apparatus to lengthen by about 0.1 mm to 3 mm. In a particular embodiment, both sequential movement phases of a particular cycle cause the limb lengthening apparatus to lengthen by about 1 mm.

FIG. 5A shows a distal portion of a lengthening apparatus in accordance with various embodiments herein. The lengthening apparatus can include a magnet 510. The magnet 510 can be toroidal in some embodiments and can include various materials as described above. The lengthening apparatus can also include a distal end cap 588 and a spring 586. The distal end cap 588 can fit within the distal end of the second body member 512 and can served to retain the spring 586 within the central lumen of the second body member 512. The second body member 512 can be made of various biocompatible materials including, but not limited to, metals, polymers, ceramics, glasses, and the like. The second body member 512 can have an outside diameter sufficient to fit within the bone of a patient, such as within the femur of a patient. The magnet 510 can be coupled, directly or indirectly, to a piston 508. The piston 508 can be made of various biocompatible materials including, but not limited to, metals, polymers, ceramics, glasses, and the like. The piston 508 can transmit the force provided by the magnet 510 when an external magnetic field is applied. The lengthening apparatus can also include locking screw conduits 582. The locking screw conduits 582, in conjunction with locking screws (not shown) can be used to affix the distal end of the lengthening apparatus within the bone of a patient.

In various embodiments, the piston 508 can include a locking screw channel 584, which can be longer than the length between the locking screw conduits 582, to accommodate movement of the piston 508 when the external magnetic field is applied with respect to the locking screw conduits 582 and screws through the second body member 512. Specifically, when the magnetic field is applied, the magnet pulls the piston 508 in the direction of arrow 583, thus compressing spring 586.

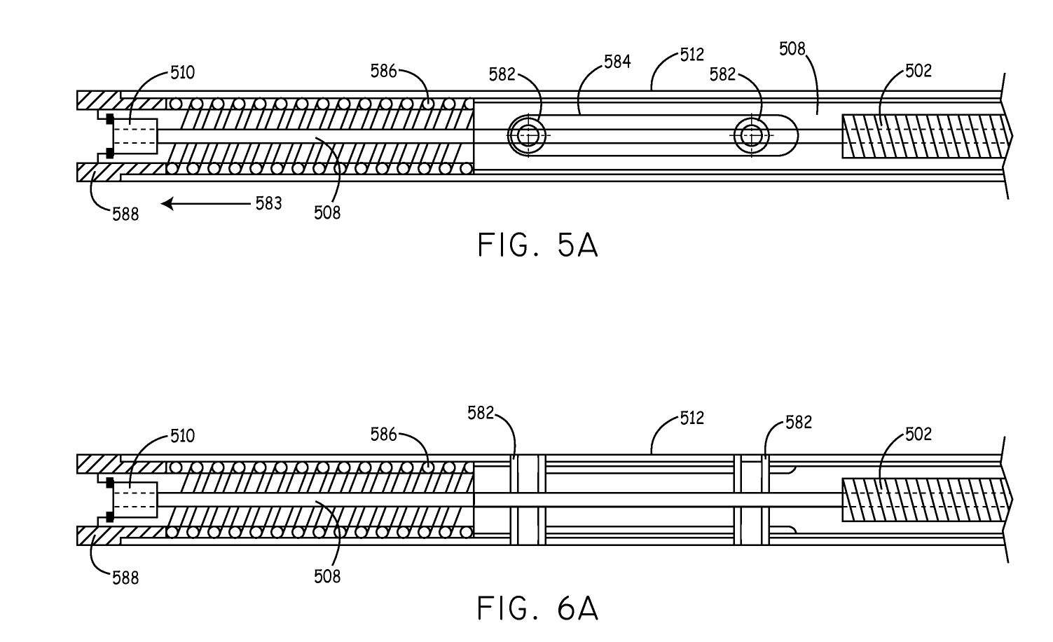

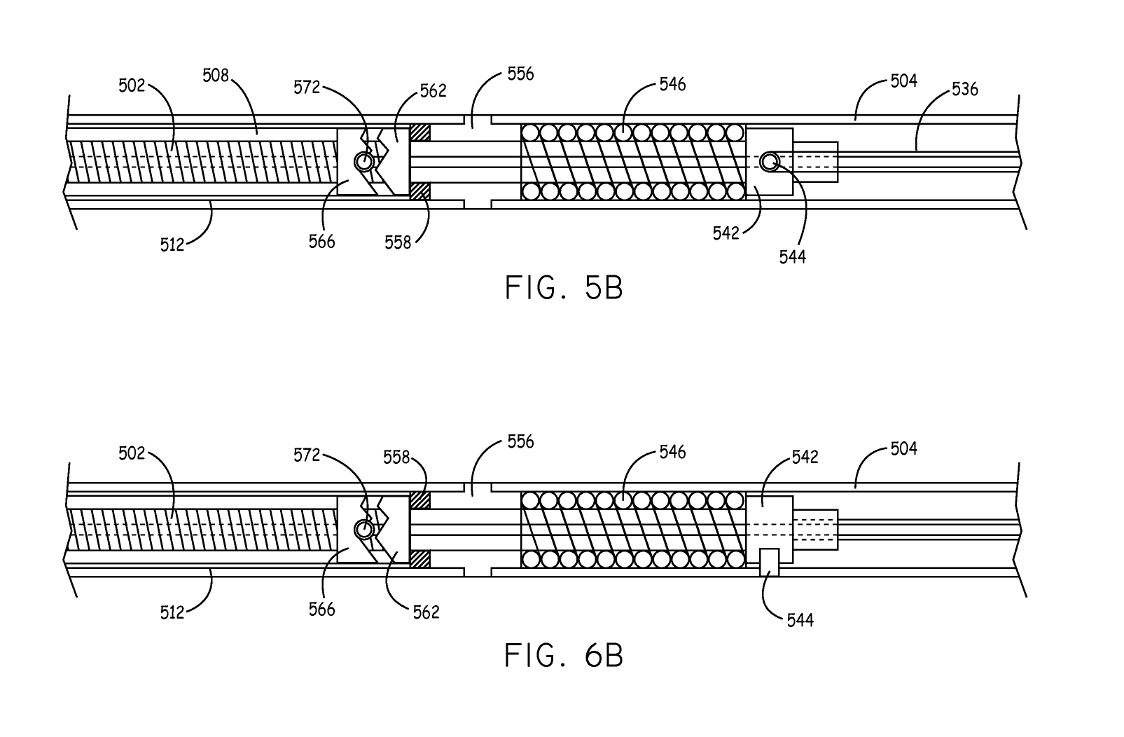

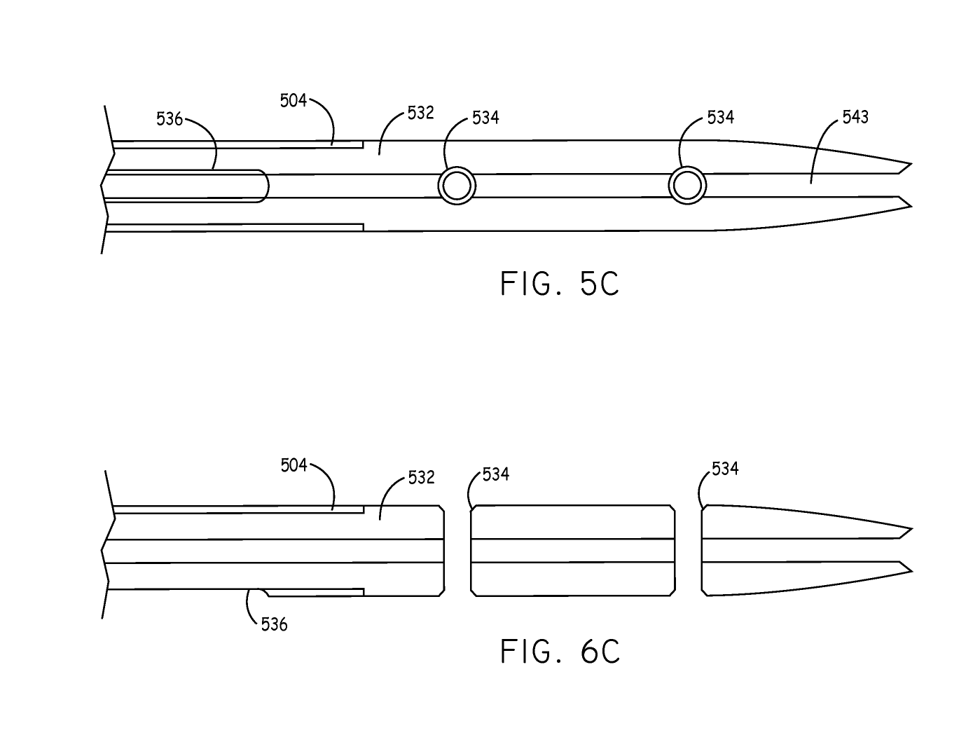

In this view, a portion of a threaded drive shaft 502 can be seen. The threaded drive shaft 502 can be made of various biocompatible materials including, but not limited to, metals, polymers, ceramics, glasses, and the like. In a particular embodiment, the threaded drive shaft 502 is made of a biocompatible metal. FIG. 6A shows the same components as FIG. 5A, but rotated 90 degrees so as to more clearly illustrate various features.

FIG. 5B shows a medial portion of a lengthening apparatus in accordance with various embodiments herein. Movement of the piston 508 and the force of spring 546 causes the first split cam half 562 to push against the drive peg 572 on the drive nut, causing the drive nut to rotate around the threaded drive shaft 502 in a manner as illustrated with respect to FIG. 4. When the external magnetic field is turned off or taken away, the force of spring 586 causes the second split cam half 566 to push against the drive peg 572 on the drive nut, causing the drive nut to rotate around the threaded drive shaft 502 and complete the second phase of the two step rotation cycle.

The thrust bearing 558 can be made of various materials. In some embodiments, the thrust bearing 558 can be made of polyethylene, such as ultra high molecular weight (UHMW) polyethylene. Junction fitting 556 can be disposed between the thrust bearing 558 and the spring 546. The spring 546 can be disposed against the proximal collar 542 at the opposite end of the junction fitting 556. An anti-rotation set screw 544 can pass through the proximal collar 542 can be disposed within an anti-rotation slot in the first body member 504, so as to prevent the threaded drive shaft 502 from rotating with respect to the first body member 504. The first body member 504 can be made of various biocompatible materials including, but not limited to, metals, polymers, ceramics, glasses, and the like. The first body member 504 can have an outside diameter similar to, or the same as, the second body member 512. FIG. 6B shows the same components as FIG. 5B, but rotated 90 degrees so as to more clearly illustrate various features.

FIG. 5C shows a proximal portion of a lengthening apparatus in accordance with various embodiments herein. In this view, a bullet 532 is shown interfacing with the first body member. The bullet 532 can be provided in variable lengths and then be fitted to the first body member so as to allow easy adjustment of the overall length of the apparatus to start so as to accommodate different long bone lengths in the limbs of patients. The proximal end of the bullet 532 can taper for easy insertion within the bone of a patient. However, in other embodiments, the bullet 532 can have different shapes. The bullet 532 can be made of various biocompatible materials including, but not limited to, metals, polymers, ceramics, glasses, and the like. The lengthening apparatus can also include locking screw conduits 534. The locking screw conduits 534, in conjunction with locking screws (not shown) can be used to affix the bullet 532 and thereby the first body member 504 within the bone of a patient. In various embodiments, one or more components of the lengthening apparatus can include a coaxial central channel 543, so as to accommodate a guide wire, or other surgical instrument. FIG. 6C shows the same components as FIG. 5C, but rotated 90 degrees so as to more clearly illustrate various features.

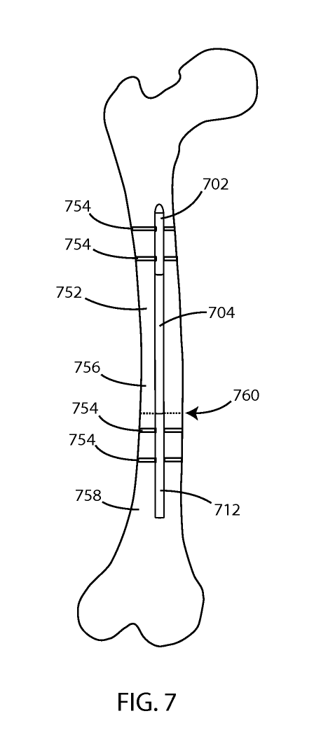

FIG. 7 shows an exemplary limb lengthening apparatus in accordance with some embodiments herein disposed within the femur 756 of a patient. The limb lengthening apparatus can include a bullet 702 (or superior-most body member), a first body member 704, and a second body member 712. In this view, the femur 756 is shown divided into a superior portion 752 and an inferior portion 758 as divided along a cut line 760. It will be appreciated that this cut line 760 is merely illustrative. The bullet 702 can be attached to the superior portion 752 of the femur with fixation screws 754. In some embodiments, the first body member 704 can be attached to the superior portion 752 of the femur with fixation screws. The second body member 712 can be attached to the inferior portion 758 of the femur 756 with fixation screws 754. As the device is actuated in order to cause the first body member 704 and the second body member 712 to move apart from one another, the callus that forms at the cut line 760 can be stretched in order to lengthen the femur 756. While a femur is shown in this illustration, it will be appreciated that apparatus included within the scope herein can be sized appropriately for placement within various bones of a patient.

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention. It should be understood that many variations and modifications may be made while remaining within the spirit and scope of the invention.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

The claims Are:

1. A limb lengthening apparatus comprising:
a magnet;
a piston having a lengthwise axis, the magnet oriented so as to cause movement of the piston along its lengthwise axis in response to application of an external magnetic field;
a first body member and a second body member, the first body member and second body member configured to be disposed lengthwise within the bone of a limb to be lengthened;
a lengthening mechanism, the mechanism comprising
a drive nut, wherein sequential application and removal of the external magnetic field causes the drive nut to rotate around the lengthwise axis of the piston causing the distance between the first body member and the second body member to increase.

2. The limb lengthening apparatus of claim 1, the drive nut comprising a central threaded lumen and a plurality of drive pegs;
the lengthening mechanism further comprising first and second split cam halves, each split cam half comprising a plurality of teeth, the teeth comprising a bearing surface that is angled with respect to the lengthwise axis of the piston;
wherein the lengthening mechanism is moveable between a first position wherein the drive pegs of the drive nut contact the teeth of the second split cam half and a second position wherein the drive pegs of the drive nut contact the teeth of the first split cam half; and
a spring exerting force on the second split cam half to bias the lengthening mechanism into the first position.

3. The limb lengthening apparatus of claim 2, wherein the teeth of the first split cam half are opposed to the teeth of second split cam half, the teeth of the first and second split cam halves comprising peaks and valleys, wherein the teeth of the first split cam half are out of phase with the teeth of the second split cam half.

4. The limb lengthening apparatus of claim 2, each split cam half comprising between two and eight teeth.

5. The limb lengthening apparatus of claim 2, wherein the bearing surface of each tooth is angled by about 10 to about 60 degrees with respect to the lengthwise axis of the piston.

6. The limb lengthening apparatus of claim 2, wherein the split cam halves cannot rotate with respect to one another.

7. The limb lengthening apparatus of claim 1, further comprising a threaded drive shaft passing through the drive nut, the drive nut configured to rotate around the threaded drive shaft.

8. The limb lengthening apparatus of claim 7, wherein sequential application and removal of the external magnetic field causes the drive nut to rotate around the lengthwise axis of the threaded drive shaft by a fixed amount causing the distance between the first body member and the second body member to increase by a fixed amount.

9. The limb lengthening apparatus of claim 8, wherein sequential application and removal of the external magnetic field causes the drive nut to rotate around the lengthwise axis of the threaded drive shaft by a fixed amount causing the distance between the first body member and the second body member to increase by approximately 1 millimeter.

10. The limb lengthening apparatus of claim 8, wherein sequential application and removal of the external magnetic field causes the drive nut to rotate around the lengthwise axis of the threaded drive shaft by about 90 degrees causing the distance between the first body member and the second body member to increase by a fixed amount.

11. The limb lengthening apparatus of claim 1, the magnet comprising a toroid magnet.

12. The limb lengthening apparatus of claim 1, the limb lengthening apparatus further comprising a third body member.

13. The limb lengthening apparatus of claim 2, further comprising a second spring exerting on the first split cam half to bias the lengthening mechanism into the second position.

14. The limb lengthening apparatus of claim 2, wherein the drive nut comprises between two and eight drive pegs.

15. The limb lengthening apparatus of claim 14, wherein the drive nut comprises four drive pegs that are spaced at 90 degree intervals around the exterior radial surface of the drive nut.

16. The limb lengthening apparatus of claim 1, wherein the magnet causes between 5 and 20 millimeters of movement of the piston along its lengthwise axis in response to application of an external magnetic field.

17. The limb lengthening apparatus of claim 1, the first and second body members comprising a biocompatible material.

18. A method of lengthening the limb of a subject, the method comprising:
implanting a limb lengthening apparatus into the subject the limb lengthening apparatus comprising:
a magnet;
a piston, the magnet oriented so as to cause movement of the piston along its lengthwise axis in response to application of an external magnetic field;
a first body member and a second body member, the first body member and second body member configured to be disposed lengthwise within the bone of a limb to be lengthened;
a lengthening mechanism, the mechanism comprising
a drive nut, wherein sequential application and removal of the external magnetic field causes the drive nut to rotate around the lengthwise axis of the piston causing the distance between the first body member and the second body member to increase;
applying an external magnetic field to the magnet; and
removing the external magnetic field to the magnet.

19. The method of claim 18, wherein the steps of applying an external magnetic field to the magnet and removing the external magnetic field to the magnet are repeated at intervals over time.

20. The method of claim 18, further comprising the step of performing a step-cut of the cortical bone in the limb to be lengthened.
